(19) United States
(12) Patent Application Publication
(10) Pub. No.: US 2011/0030730 A1
(43) Pub. Date: Feb. 10, 2011

(54) SYSTEM FOR PRODUCING AND DISTRIBUTING AN OZONATED FLUID

(76) Inventor: Daniel W. Lynn, Omaha, NE (US)

Correspondence Address: POLSINELLI SHUGHART PC, 700 W. 47TH STREET, SUITE 1000, KANSAS CITY, MO, 64112-1802, US

(21) Appl. No.: 12/816,861

(22) Filed: Jun. 16, 2010

Related U.S. Application Data
(63) Continuation-in-part of application No. 12/047,442, filed on Mar. 13, 2008.

(51) Int. Cl. B08B 3/04; B01F 3/04; A61L 2/16

(52) U.S. Cl. 134/18; 134/115 R; 134/113; 134/57 R; 134/36

(57) ABSTRACT

A system for producing and distributing an ozonated fluid is described. The system includes a tank for a fluid. A skid is in fluidic communication with the tank to receive the fluid from the tank. The skid includes an ozone generator to generate ozone gas and an injector to inject the fluid with the ozone gas to produce an ozonated fluid. A distribution network distributes the ozonated fluid for application. The distribution network is in fluid communication with the tank to return unapplied ozonated fluid to the tank.

### CROSS REFERENCE TO RELATED APPLICATION

This application is a continuation-in-part of U.S. Nonprovisional patent application Ser. No. 12/047,442 filed Mar. 13, 2008, which is hereby incorporated by reference.

### FIELD OF INVENTION

The present invention relates to a system for producing and distributing an ozonated fluid for use in cleaning and sanitation.

### BACKGROUND OF INVENTION

Ozone in a solution has been previously used for cleaning and sanitizing. Maintaining a solution with a consistent ozone concentration has proven difficult. Producing large quantities of ozone solution has also proven difficult. Ozone is unstable, which provides for it cleaning and sanitizing capabilities, but also makes consistent ozone levels difficult to maintain in a solution. If the ozone solution has too much ozone or large gas bubbles of ozone, then off-gassing problems may occur, as the excess ozone is released into the work facility creating environmental problems and possibly violating workplace safety regulations. If the solution has too little ozone, then the cleaning and sterilizing may not be as effective as desired. Ozone solutions have proven difficult to consistently and uniformly prepare in sufficient quantities required for industrial cleaning applications. Ozone gas cannot be packaged or stored and must be generated on site.

### SUMMARY OF INVENTION

A system for producing and distributing an ozonated fluid is described herein. The system prepares ozonated fluid for use in cleaning and sanitation. The system supplies the ozonated fluid to a distribution network and/or a tank. The distribution network supplies applicators with the ozonated fluid for application. The distribution network further returns unapplied ozonated fluid back to the tank. As such, the ozonated fluid is reused or recycled by the system. The system selectively and variably directs the ozonated fluid produced by the system to either the distribution network for application and/or to the tank for preparing a batch of ozonated fluid.

The system may include an optional applied dosage monitoring system to measure ozone concentration with a remote sensor in the ozonated fluid at a point of application and modulate the concentration of the ozonated fluid at production. The system may also include an optional OSHA compliance package, which monitors ozone gas levels in the ambient air at the point of application and stops distribution of the ozonated fluid if ozone gas levels exceed designated levels.

The use of ozonated fluid provides many advantages, including the elimination of harmful pathogens. Oxygen and purified water are the only by-products of producing and cleaning with the ozonated fluid. The use of ozonated fluid reduces the hydraulic load on waste water treatment systems. Ozonated fluid destroys known pathogens that have developed resistance to standard cleaning and sanitizing methods. For example, ozonated fluid has proven effective against salmonella, e. coli, MRSA, and campylobacter. The use of ozone as a cleaning and sterilizing agent is a chemical treatment like other oxidizers, including chlorine, potassium permanganate, hydrogen peroxide, etc. without the disadvantages of said chemicals. Ozonated fluid is effective even when applied at low pressure and with cold water. Ozonated fluid may be used during production, thus eliminating machinery down time, reducing employee costs and increasing production output.

Ozonated fluid is safe and easy to use. Unlike harsh, dangerous chemicals, the system is safe for employees and does not require the extensive employee protection necessary with traditional sanitation processes. The system provides ozonated fluid on-demand and on-site without chemicals. The system eliminates the expense and danger of transporting and storing hazardous sanitation supplies.

The system converts ozone gas into a more stable and long-lasting form for more effective sanitization processes. The system processes the ozonated fluid to reduce bubble size.

The system reduces energy costs. Instead of using hot water as needed with traditional sanitation systems, the system uses cold water, thereby reducing energy costs. The system further provides reduced costs associated with residual water on waste water treatment systems.

The system is modular and may be installed anywhere throughout a facility or the production process. Unlike traditional sanitation processes that require the complete shutdown of machinery, the system can apply the ozonated fluid during the production process and directly to food. Ozonated fluid may be applied on all hard equipment surfaces. The system may be installed to allow for continuous sanitation without shutting down machinery. The system is a chemical-free system that destroys the biofilm on hard surfaces during food processing and production in food processing and other facilities. The system allows for continuous or extended production in the facility. When installed in processing facilities, the hard surfaces can be maintained 24 hours a day, 7 days a week accomplishing both a microbial reduction as well as improving aesthetics.

The ozonated fluid may be applied to areas that receive and process live animals or animal parts, directly to food (FDA approved for direct to food contact) and used for air decontamination and odor control throughout plant or in specific areas. The ozonated fluid may be used in food chillers to help extend shelf-life of product without sacrificing taste or quality and vehicles used to transport live animals. The ozonated fluid may be used in all drains, floor, walls, break rooms, restrooms or public areas. The ozonated fluid may be used on processing equipment, floors, tables, etc. The ozonated fluid may be applied at a high pressure to the hard surfaces, and is effective for sanitizing the hard surfaces and removing soils and bulk materials from the hard surfaces.

The system provides up to approximately 50 gallons per minute of ozonated fluid. The system is modular. As such, the system may be disassembled and moved about a plant or facility to different locations. The system supplies the distribution network with the ozonated fluid. The system includes a skid to prepare the ozonated fluid.

The distribution network is in fluid communication with the skid to receive the ozonated fluid. The distribution network is arranged about a facility to distribute the ozonated fluid to any of a variety of applicators and/or auxiliary lines that further distribute the ozonated fluid to other applicators. The applicators may include, for example, sprayers, wands, faucets, hoses and other devices commonly used for spraying or discharging fluids.

The tank holds a reservoir of fluid, such as water. The tank is supplied with fresh water by a water supply line in fluid communication with a water supply, such as municipal water supply. A tank fill line fluidly connects the skid with the tank. The tank fill line supplies fluid from the tank to the skid, where the skid ozonates the fluid. The ozonated fluid is then outputted to the distribution network. A distribution network return line directs ozonated fluid from the distribution network back to the tank. As such, unused ozonated fluid from the distribution network may be returned to the tank.

An ozonated fluid supply line is in fluid communication with the tank and the skid. The supply line directs ozonated fluid produced in the skid to the tank. This allows the tank to be filled with a batch of ozonated fluid. Such a batch may be prepared for use in conjunction with the ozonated fluid prepared by the skid. In certain embodiments, the tank may further be in fluidic communication with an optional application pump. The application pump may be used to distribute an ozonated fluid that is prepared as a batch in the tank.

In one aspect, a system for producing and distributing an ozonated fluid is provided. The system includes a tank for a fluid. A skid is in fluidic communication with the tank to receive the fluid from the tank. The skid includes an ozone generator to generate ozone gas and an injector to inject the fluid with the ozone gas to produce an ozonated fluid. A skid output line is in fluidic communication with the skid and a distribution network to supply the distribution network with the ozonated fluid. The skid output line is also in fluidic communication with the skid and the tank to supply the tank with the ozonated fluid. The distribution network distributes the ozonated fluid for application. The distribution network is in fluid communication with the tank to return unapplied ozonated fluid to the tank.

In another aspect, a system for producing and distributing an ozonated fluid is provided. The system includes a tank to store a fluid. The system includes a skid to produce ozonated fluid. A skid supply line fluidly connects the tank with the skid to supply the skid with the fluid. The skid includes an ozone generator to generate ozone gas and an injector to inject the fluid with the ozone gas to produce an ozonated fluid from the fluid. A skid output line outputs the ozonated fluid from the skid. One or more fluid lines connect to the skid output line that receive the ozonated fluid from the skid. The one or more fluid lines include a first valve to open or close the one or more fluid lines. The one or more fluid lines supply one or more applicators with the ozonated fluid for application. A tank fill line fluidly connects to the tank and the skid output line to supply the tank with ozonated fluid. The tank fill line includes a second valve to open or close the tank fill line.

In another aspect, a system for producing and distributing an ozonated fluid is provided. The system includes a reservoir for a fluid. A skid is in fluidic communication with the reservoir to receive the fluid from the reservoir. An inlet pump supplies the skid with the fluid. The skid includes an oxygen concentrator to produce oxygen gas, and the oxygen concentrator is in supply communication with an ozone generator to generate ozone gas from the oxygen gas. An injector pump supplies an injector with the fluid from the inlet pump. The injector injects the fluid with the ozone gas from the ozone generator to produce an ozonated fluid. A degassing system removes excess ozone gas from the ozonated fluid. An ozone destruct unit destroys the excess ozone gas. A reaction vessel processes the ozonated fluid. A skid output line includes valves for selectively supplying the ozonated fluid to a distribution network or to the reservoir. The distribution network distributes the ozonated fluid to one or more applicators that spray or apply the ozonated fluid.

In another aspect, a method for producing and distributing an ozonated fluid is provided. The method includes providing a reservoir for a fluid and a skid in fluidic communication with the reservoir to receive the fluid from the reservoir. The skid includes an ozone generator to generate ozone gas and an injector to inject the fluid with ozone gas to produce an ozonated fluid. A distribution network distributes the ozonated fluid for application. The distribution network is in fluid communication with the reservoir to return ozonated fluid to the reservoir. The method further includes providing fluid to the reservoir and pumping the fluid from the reservoir to the skid. The method further includes ozonating the fluid at the skid to produce an ozonated fluid and distributing the ozonated fluid via the distribution network. The method further includes applying a first portion of the ozonated fluid and returning a second portion of the ozonated fluid to the reservoir.

In another aspect, a system to measure ozone gas levels in ambient air for use with ozonated fluid dispensing equipment is provided. The system includes hosing, which includes a collection opening to receive sample air. An ozone sensor measures ozone levels in the sample air. A pump is in fluidic communication with the hosing to transfer the sample air to from the collection opening to the ozone sensor, and the ozone sensor measures the ozone levels in the sample air.

In another aspect, a system to monitor ozone levels of an ozonated fluid applied by ozonated fluid producing and dispensing equipment is provided. The system includes a local sensor positioned to measure dissolved ozone levels in ozonated fluid produced by equipment for producing and dispensing ozonated fluid. A remote sensor is positioned at point of application of the ozonated fluid to measure dissolved ozone levels in the ozonated fluid at the point of application. The local sensor is in electrical communication with a local monitor. The remote sensor in electrical communication with a remote monitor.

### DESCRIPTION OF FIGURES

FIG. 1 is a schematic representation of the system for producing and distributing an ozonated fluid.

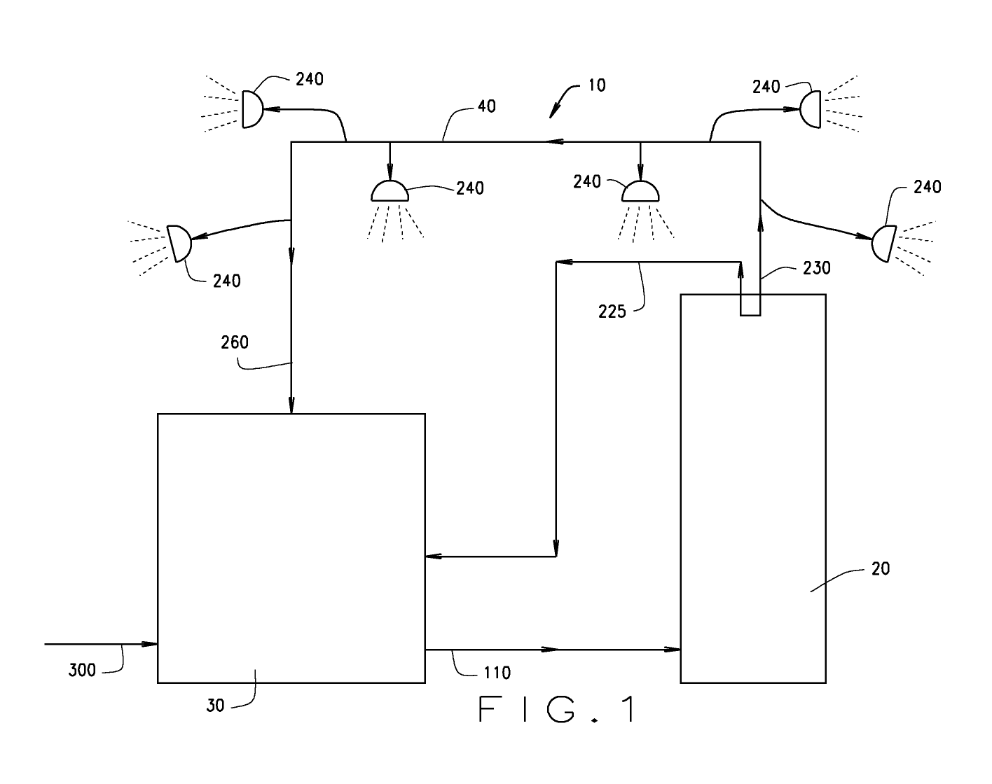

FIG. 2 is a further schematic representation of the system for producing and distributing an ozonated fluid.

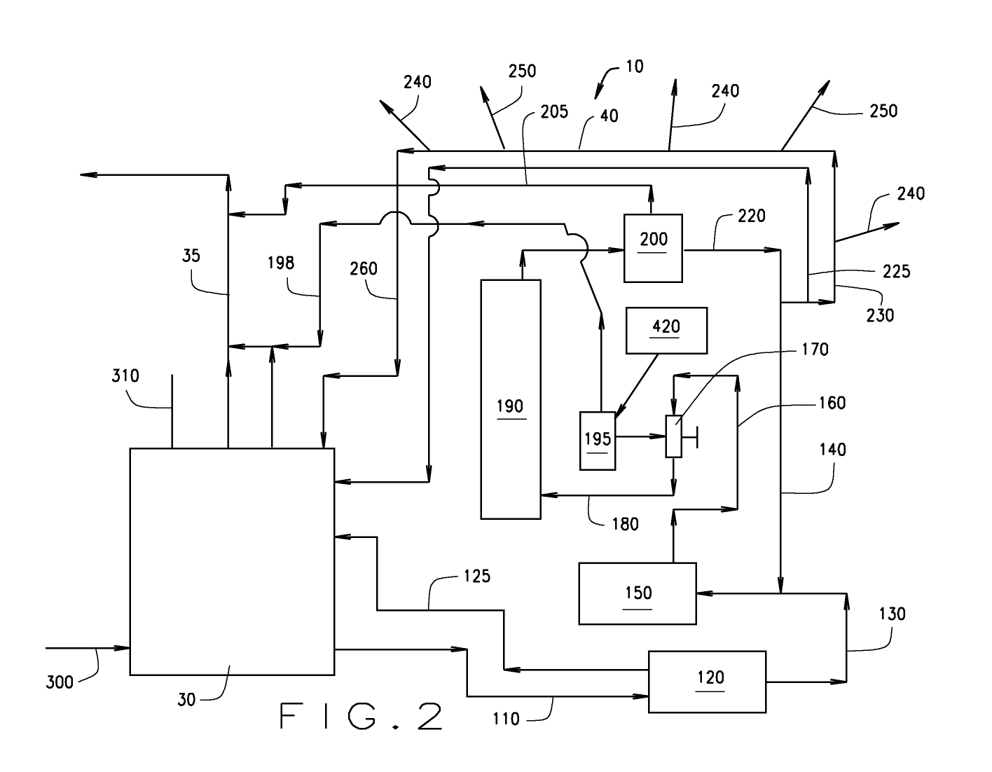

FIG. 3 is a rear view of the skid for producing the ozonated fluid.

FIG. 4 is a front view of the skid for producing the ozonated fluid.

FIG. 5 is a sectional view of the reaction vessel.

FIG. 6 is a view of the applied dosage monitoring system.

FIG. 7 is a view of the OSHA compliance package.

FIG. 8 is a view of the system for producing and distributing an ozonated fluid with an optional application pump.

### DETAILED DESCRIPTION OF INVENTION

A system 10 for producing and distributing an ozonated fluid will now be described with reference to the FIGS. The system 10 produces an ozonated fluid, such as an aqueous ozone solution, from a central location in an industrial facility and distributes the ozonated fluid via a distribution network 40 to different application points throughout the industrial facility. The system 10 produces the ozonated fluid to attack and destroy pathogens and act as a no-rinse sanitizer for hard surfaces in a variety of applications, especially industrial processing facilities related to food processing.

The system 10 includes a skid 20, a tank 30, and the distribution network 40. The tank 30 supplies the skid 20 with fluid, such as water, for ozonation. The skid 20 prepares the ozonated fluid from the fluid received from the tank 30. The distribution network 40 distributes the ozonated fluid for application. The tank 30 holds the supply water or supply fluid for skid 20. Pumps direct the ozonated fluid throughout the facility.

The tank 30 holds the water and fluid to supply the skid 20, where the water or the fluid is ozonated. A tank water supply line 300 is in fluidic communication with the tank 30 to fill the tank 30 from a municipal water supply. The tank 30 may hold approximately 200 gallons to approximately 1000 gallons of fluid. The embodiment illustrated in the FIGS. uses a 400 gallon tank. Other versions or embodiments of the system 10 may include larger or smaller tanks 30.

The tank 30 provides several advantages. The tank 30 provides a reservoir of fluid to supply the skid 20 that is not affected by fluctuations in demand, seasonal changes, or the like. As such, the tank 30 provides the skid 20 with a consistent supply of fluid for ozonation. Also, the tank 30 may be used to store and received ozonated fluid produced by the skid 20 when the operator desires a batch of ozonated fluid to be ready for immediate application. The operator may program or direct the system 10 to fill the tank 30 with the ozonated fluid instead of supplying the distribution network 40 with the ozonated fluid. Thus, the tank 30 may be filled or nearly filled with ozonated fluid produced by the skid 20. The tank 30 further provides a receptacle to receive excess ozonated fluid from the distribution network 40. Ozonated fluid that is not applied by the distribution network 40 may be returned to the tank 30.

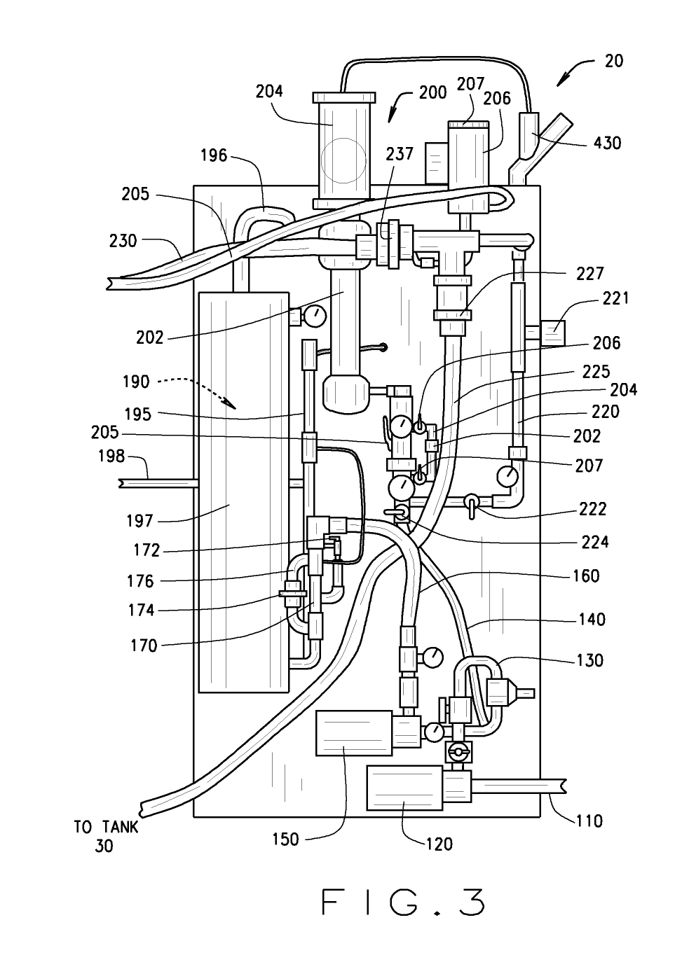

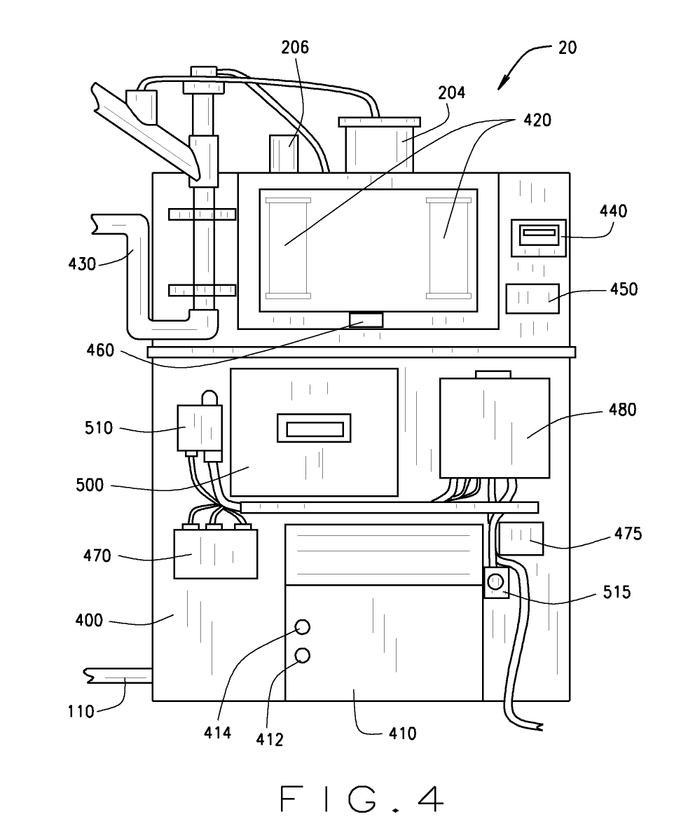

The system 10 will now be described with reference to FIGS. 3-4, which show the front (FIG. 3) and the rear (FIG. 4) of the skid 20. A skid supply line 110 is in fluidic communication with the tank 30 and an inlet pump 120 of the skid 20, which transfers fluid from the tank 30 to the skid 20. The skid supply line 110 thus supplies the skid 20 with the fluid for ozonation.

The inlet pump 120 is in fluidic communication with an injector pump 150 via an injector pump line 130. The injector pump line 130 supplies the injector pump 150 with the fluid from the inlet pump 120. A skid return line 140 is also in fluidic communication with the injector pump line 130. Ozonated fluid that is not sprayed or applied by the distribution network 40 or that does not enter the distribution network 40 is returned to the injector pump line 130 via the skid return line 140 and is reused in the system 10. As such, the system 10 forms a loop for the distribution of the ozonated fluid.

The injector pump 150 supplies pressure to the fluid to direct the fluid via an injector line 160 to an injector 170. A suitable pump for the injector pump 150 and the inlet pump 120 are a 1 and ½ HP pump.

The injector 170 also receives ozone gas prepared by an ozone generator 420. The injector 170 injects the ozone gas into the fluid from the injector line 160. The injector 170 may include a mazzei injector or other type of venturi to mix the ozone gas with the water. Any of a variety of injectors could be utilized. The injector 170 creates a vacuum to draw the ozone gas from the ozone generator 420 and then dissolves the ozone in the fluid from the injector line 160. An injector pressure gauge 172 may determine the suction level of the injector 170. An injector control valve 174 is used to increase or decrease the suction pressure on the injector 170 by opening or closing a by-pass 176 around the injector 170.

The injector 170 outputs the ozonated fluid into a reaction vessel line 180 which directs the fluid to a reaction vessel 190. The reaction vessel 190 further processes the ozonated fluid. The reaction vessel 190 may be contained or housed in a reaction vessel tank 197.

The reaction vessel 190 further processes the ozonated fluid to reduce the bubble size of the ozone gas in the ozonated fluid. The reaction vessel 190 further reduces the number of ozone gas bubbles in the ozonated fluid to increase the concentration of ozone in the ozonated fluid. Breaking down the bubbles of ozone into smaller bubbles of ozone increases the oxidation reduction potential of the ozone in the aqueous ozone solution. The greater oxidation reduction potential of the ozonated fluid allows the ozone to act not only as a sanitizer, but as a degreaser and therefore has more oxidizing power than conventionally mixed solutions. Decreasing the bubble size of the ozone gas also assists in maintaining a uniform concentration of ozone gas in the ozonated fluid.

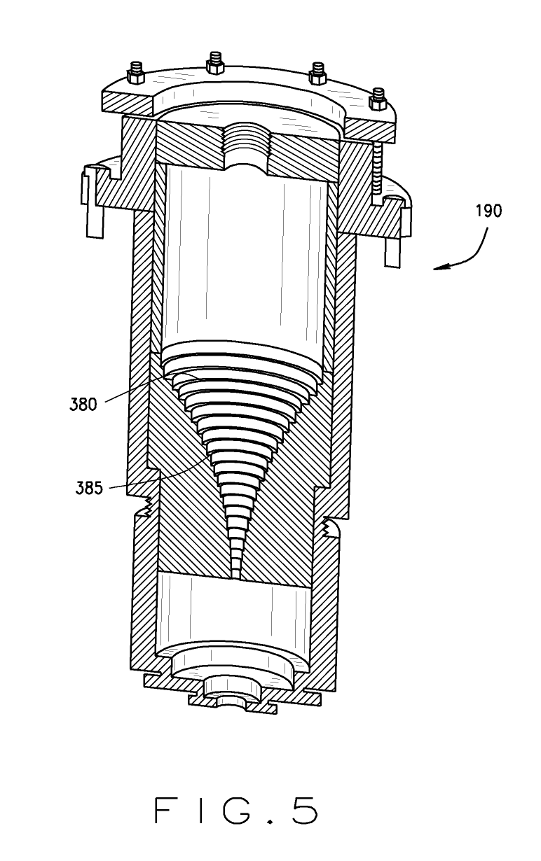

A suitable reaction vessel 190 is shown in FIG. 5. The reaction vessel 190 is further described in U.S. Patent Application Publication No. 2009/0008806, which is hereby incorporated by reference herein in its entirety. Other types of vessels and processors to process the ozonated fluid may be used with the system 10. The reaction vessel 190 comprises a conical-shaped surface 385 having a plurality of edges 380 on the conical-shaped surface 385. The conical-shaped surface 385 imparts a rotating action or a vortex to the ozonated fluid entering the reaction vessel 190 from the reaction vessel line 180, and the ozonated fluid rotates about the conical-shaped surface 385.

From the reaction vessel 190, the fluid exits the reaction vessel 190 via an ozone degassing line 196 and is passed to an ozone degassing system 200, which includes a degas separator 202 to remove the excess ozone gas from the ozonated fluid. The degas separator 202 removes the excess ozone bubbles from the ozonated fluid to reduce the levels of free ozone gas released at an application point during the spraying of the ozonated fluid, which in high concentrations could breach OSHA regulations. The degas separator 202 includes a degas valve 204. Ozone enters from the bottom of the degas valve 204 and bubbles up through the degas valve 204 and out the top of the degas valve 204.

The ozone gas from the degas valve 204 is passed to the ozone destruct unit 206, which holds a catalyst to destruct the extra ozone gas. Ozone destruct heat tape 207 is positioned on the ozone destruct unit 206 to keep the catalyst dry and functioning. The ozone destruct unit 206 turns the ozone gas back into oxygen gas. The ozone destruct unit 206 vents waste gas via an ozone destruct vent line 205, such as 1″ braided tube, to the tank vent line 35. A suitable ozone destruct unit is commercially available as Model Number 4WM from Ozone Water Systems of Phoenix, Ariz.

From the ozone degassing system unit 200, the ozonated fluid passes to a skid output line 220. The skid output line 220 includes a dissolved ozone monitor probe 202 that is positioned in a probe loop 204. The probe 202 checks the ozone level of the fluid in the probe loop 204. A by-pass valve 205 is used to force solution into the probe loop 204. A top probe valve 206 closes off a top portion of the probe loop 204. A bottom probe valve 207 closes off the bottom portion of the probe loop 204.

The skid output line 220 includes a shut off valve 222 to stop solution from leaving the skid 20. A recirculation valve 224 in the skid return line 140 is used to direct the solution back though the skid 20.

The skid output line 220 branches into a tank fill line 225 and a distribution network supply line 230. The tank fill line 225 includes a tank valve 227 to direct the solution to the tank 30 and to open and close the tank fill line 225. The distribution network supply line 230 includes a distribution network valve 237 to direct solution to distribution network 40 and to open and close the distribution network supply line 230. As such, the skid output line 220 may selectively direct the ozonated fluid to the tank fill line 225, in order to fill the tank 30, and to the distribution network supply line 230, in order supply the distribution network 40. The skid output line 220 may send different portions or percentages of the output of ozonated fluid of the skid 20 to the tank 30, via tank the fill line 225, and to the distribution network supply line 230, via the distribution network supply line 230, by adjusting the valves 227 and 237. The valves 227 and 237 may be fully opened, fully closed, or variably opened in a range of between 0% open and 100% open in order to control and modulate the flow of the ozonated fluid to the tank 30 and/or the distribution network 40. For example, the tank 30 may receive 25% of the ozonated fluid outputted by the skid 20, while the distribution network 40 may receive 75% of the ozonated fluid outputted by the skid 20, and vice versa. Of course, the skid output line 220 may also send all of the output of the skid to either the tank 30 or the distribution network 40.

As such, by closing the distribution network valve 237, the ozonated fluid passes back to the tank 30 via the tank fill line 225 in order to prepare a large, ready to use batch of ozonated fluid that is stored in the tank 30. The batch may fill a portion or the entire volume of the tank 30. Also, by closing the tank valve 227, the ozonated fluid may pass to the distribution network supply line 230, which is used to direct the ozonated fluid about a facility or other area for application of the ozonated fluid. The distribution network supply line 230 includes the distribution network valve 237 to direct the solution to the distribution network 40 and to open and close the distribution network 40.

The distribution network 40 may include lines, such as hosing, tubing, piping, or other conduits that distribute the ozonated fluid about a facility or other environment. The lines of the distribution network 40 and the system 10 may include plastic, rubber, metal, braided materials to transfer the fluid ranging in diameter from approximately ¼ inch to approximately 6 inches or more. The distribution network 40 may include hundreds or thousands of feet of lines that are in fluidic connection with the skid 20 to distribute the ozonated fluid. The distribution network 40 forms a circuit or a loop that fluidly connects the tank 30 and the skid 20. Typically, the fluid is drawn from the tank 30, the fluid is ozonated at the skid 20, and the ozonated fluid is applied at various locations by the distribution network 40.

The distribution network 40 may include a variety of applicators 240 or and/or auxiliary networks 250 that branch off and further distribute the ozonated fluid about the facility or other environment. The applicators 240 may include, for example, sprayers, wands, faucets, hoses, dispensers, and other devices commonly used for spraying or discharging fluids. The applicators 240 may, for example, be positioned over a conveyor belt or food preparation surfaces, in kitchens and bathrooms, at wash stations, etc. in order to clean, sanitize, disinfect, etc. Additional or auxiliary pumps may added to the distribution network 40 in order to further disseminate the ozonated fluid about the facility or other environment. The distribution network 40 terminates in a distribution network return line 260 that returns unused ozonated fluid back to the tank 30. At the tank 30, the unused or unapplied ozonated fluid may be re-ozonated and passed again through the skid 20.

The tank 30 includes a tank vent line 35 to vent excess gas from the tank 30 to the atmosphere. The tank 30 further receives a vacuum break vent line 198 to receive gas from a vacuum break 195. The tank 30 further receives a pressure relief line 125 to receive pressurized fluid or gas from the inlet pump 120. The tank 30 further includes a dissolved ozone monitor line 310 connecting to a dissolved ozone sensor in the tank 30 that detects and senses the ozone levels of the fluid in the tank 30. The dissolved ozone monitor line 310 is in electrical communication with the control processor 500.

The controls and components on a front 400 of the skid 20 will now be described with reference to FIG. 4. The skid 20 includes an oxygen concentrator 410 that prepares oxygen gas from ambient air. The oxygen concentrator 410 is in communication with the ozone generator 420. In certain embodiments, the oxygen concentrator 410 provides approximately 6 CFH of oxygen gas at 10 psi. A suitable oxygen concentrator for the oxygen concentrator 410 is commercially available from the AirSep Corporation as the TOPAZ or TOPAZ PLUS and utilizes pressure swing adsorption to produce oxygen at a flow of 12-17 scf/hr at a purity of approximately 93%. The oxygen concentrator uses compressed air from its internal compressor as a feed gas to produce oxygen. Ambient air enters the intake of the oxygen concentrator and flows to the into the air compressor, which pressurized the feed air and delivers the feeds air to a heat exchanger for cooling. The cooled pressurized air then enters one adsorber, while another adsorber exhausts oxygen gas.

The oxygen concentrator 410 supplies the oxygen gas to the ozone generator 420. The ozone generator 420 uses corona discharge to make the ozone gas that is directed to the injector 170. Some suitable ozone generators 420 operate at 4500 volts DC. Some suitable ozone generators include Models CD1500p and CD2000P commercially available from ClearWater Tech, LLC of San Luis Obispo, Calif. Such ozone generators provide high concentrations of ozone gas (up to approximately 10%) at 10 PSI. The ozone generators pass the oxygen gas through a high voltage electrical filed to form single oxygen atoms which recombine to form the ozone gas. One or more ozone generators 420 may be utilized by the system 10.

An ozone Gas P-Trap 430 is used to separate moisture from the ozone gas headed to the ozone destruct unit 206. A local dissolved ozone monitor 440 monitors and displays for the level of ozone in the ozonated fluid produced by the skid 20. The control processor 500 receives the measurements from the local dissolved ozone monitor 440 and adjusts the concentration of ozone in the ozonated fluid as needed. For example, the control processor 500 may increase the output of the ozone generator 420 to increases the flow of ozone directed to the injector 170.

A power supply box 450 provides an electrical source for the dissolved ozone monitor 440, the ozone generator 420, and the oxygen concentrator 410. An on/off switch 460 for the ozone generator 420 is positioned on the front of the skid 20. An ambient ozone monitor 470 monitors the level of ozone in the ambient air. The ambient ozone monitor 470 includes a display or readout of the monitored levels. An ambient ozone analyzer 475 samples the air for ozone gas and provides measured readings to the ambient ozone monitor 470.

A breaker box 480 for the main electrical power source is provided. The control processor 500, with a touch screen, is used to monitor and control the operations of the skid 20. The control processor 500 may include one or more microprocessors, computers, and peripherals to operate the system 10. A CFH gauge 412 determines the volume of air movement from the oxygen concentrator 410.

A skid pump on/off switch 510 starts and stops the injector pump 150. An inlet pump on/off switch 515 starts and stops the inlet pump 110. An oxygen concentrator on/off switch 414 controls the oxygen concentrators 410.

In order to operate the system 10, first, all valves should be in the open position to allow water to flow to the system 10. Next, the ozone destruct switch on top of the system 10 is turned on. Next, the inlet water pump 110 is turned on. Then, the injector pump 150 is turned on. After the water is flowing, the oxygen concentrator 410 is turned on. Finally, the ozone generator 420 is turned on. In order to turn off the system 10, the equipment is turned off in the reverse order.

The system 10 produces up to approximately 50 gallons per minute of ozonated fluid having an ozone concentration of up to approximately 5 parts per million. The system 10 may produce an ozonated fluid with concentrations of ozone greater than 5 parts per million by reducing or restricting flow through the system 10. For example, the system 10 may produce up to approximately 25 gallons per minute of an ozonated fluid having an ozone concentration of up to approximately 10 parts per million. For example, the system 10 may produce up to approximately 5 gallons per minute of an ozonated fluid having an ozone concentration of up to approximately 20 parts per million.

The distribution network 40 may form a recirculation loop. The recirculation loop reduces problems associated with changes in demand, as any unused ozonated fluid is returned to the reservoir.

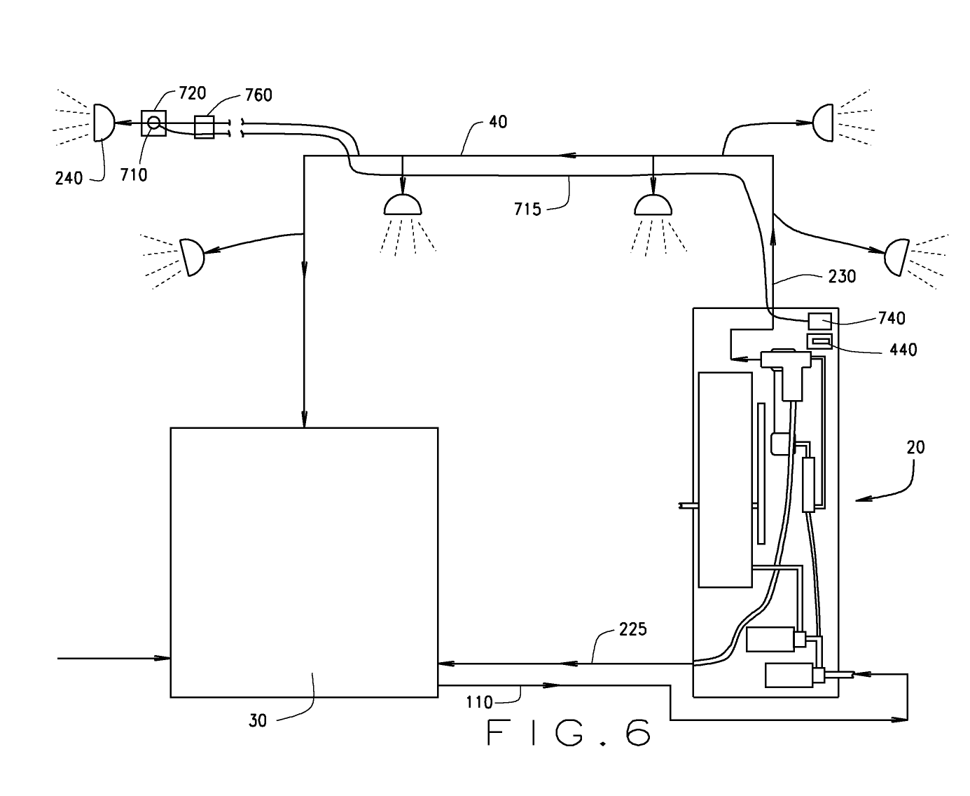

As shown in FIG. 6, in certain embodiments, the system 10 includes an optional applied dosage monitoring system 700. The control processor 500 modulates the concentration of ozone in the ozonated fluid produced by the system 10 based on the applied dosage monitoring system 700. The applied dosage monitoring system 700 measures ozone concentration with a remote sensor in the ozonated fluid at the point of application. The ozone levels may vary at the application point due to the weather, humidity time, flow rates, etc. and due to the inherent characteristics of ozone that cause it to rapidly decay. The control processor 500 is set to a specific ORP and maintains this specific ORP level for the ozonated fluid. The desired ORP level for the ozonated fluid is inputted into control processor 500. The local dissolved ozone monitor 440 measures the amount of ozone in the ozonated fluid at the system 10, while a remote probe measures the amount of ozone in the ozonated fluid actually at the applicator 240. The applied dosage monitoring system 700 determines when the system 10 needs to adjust the ozone levels based on measurements taken by the remote probe at the point of application.

The remote probe is placed in the or fluidly connected to the distribution network 40 at the point of application of the ozonated fluid, which may be several hundred feet from the skid 20. The remote probe may include a dissolved ozone sensor 710 placed at the furthest point in the distribution network 40 that the system 10 is applying the ozonated fluid. The sensor 710 is in electrical communication with a remote monitor 740 to display the measurements obtained by the sensor 710. The remote monitor 740 is typically positioned at the system 10 and is in electrical communication with the sensor 710 via a communication line 715. Typically, the sensor 710 will be positioned in hosing, tubing, piping, etc. that is supplying the applicator 240 with ozonated fluid. This provides a critical control point to measure the ORP of the applied ozonated fluid.

A suitable monitor/sensor is commercially available from the from Analytical Technology, Inc. of Collegeville, Pa. as Model Q45H/64, which uses a polarographic membraned sensor to measure dissolved ozone levels in the ozonated fluid. The sensor 710 is incorporated into a flow cell 720. The flow cell 720 is fluidly tied into the distribution network 40. The flow cell 720 may be positioned in the distribution network 40 just before or immediately prior to the ozonated fluid reaching the applicator 240. The sensor 710 is in electrical communication with the remote monitor 740. An optional junction box 760, may be used to boost the amperage of the signal from the sensor to the monitor, especially when the sensor is beyond 100 feet or so from the system 10. A suitable junction box is commercially available from ATI as Model Q15M.

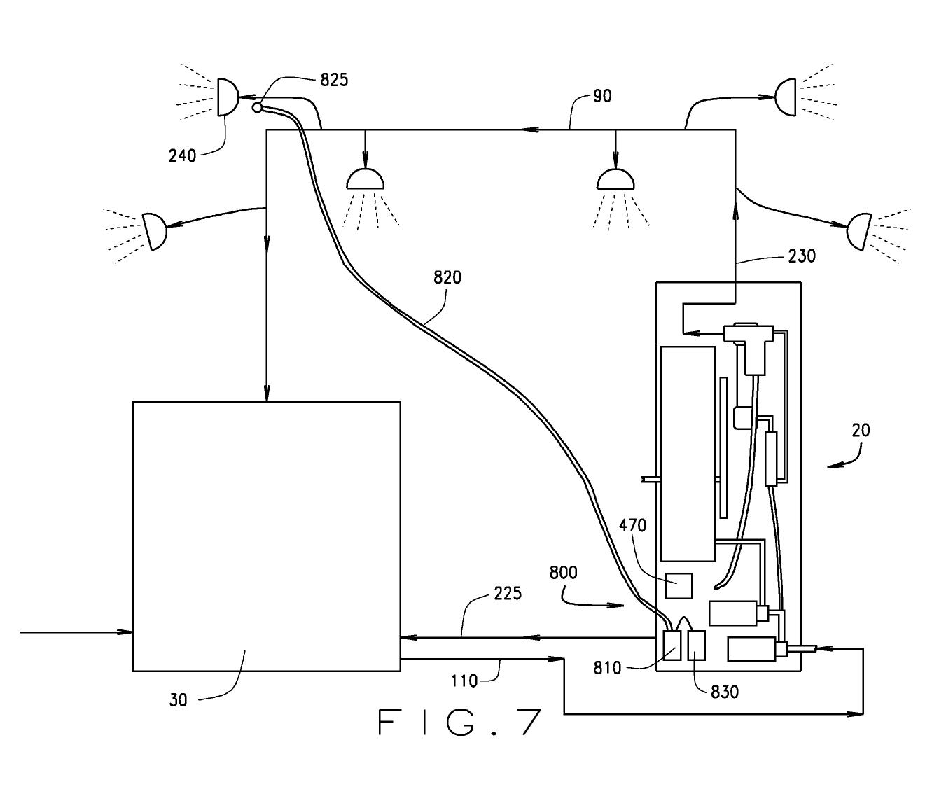

As shown in FIG. 7, in certain embodiments, the system 10 includes an optional OSHA compliance package, which monitors ozone gas levels in the ambient air at a point of application and stops distribution of the ozonated fluid if ozone gas levels exceed designated levels.

The OSHA compliance package may be used with the system 10 or other systems that generate ozonated fluid or ozone gas. OSHA currently recommends limiting human exposure to ozone levels of greater than 0.1 ppm. The OSHA compliance package measures ozone levels constantly or regularly and shuts down the system 10 if the ozone levels exceed specific levels. The OSHA compliance package may shut the system 10 down by cutting electrical power to the system 10. An ambient air ozone probe is placed at the point of application of the ozonated fluid. The ambient air ozone probe collects samples of the ambient air. An ambient air ozone sensor measures the levels of ozone in the samples of air. If the air samples contain too high a level of ozone gas, then the system 10 or other system is shut down.

An example of an OSHA compliance package 800 is shown in FIG. 7. The compliance package 800 includes a pump 810, hosing 820, an ozone sensor 830. The compliance package may be used in facilities with ozone based cleaning systems, such as the system 10 described herein, or any of the systems described in U.S. Patent Application Publication No. 2009/0120473, which is hereby incorporated by reference in its entirety, or other ozone generating systems. The compliance package 800 is in electrical or control communication with the control processor 500 of the system 10. The level of ozone that causes the system 10 to shut down may be programmed or changed by the operator. Current OSHA regulations recommend that ozone gas levels in the ambient air not exceed 0.1 ppm. The compliance package may include an electrical controller that signals or initiate the shut-down of the system 10 when the ozone levels measured by the ozone sensor 830 exceed the threshold level.

The hosing 820 extends to a collection point in the facility remote from the system 10 or other ozonated liquid/gas generating system. The hosing 820 includes a collection opening 825. The hosing 820 is in fluidic communication with the pump 810, such that the pump 810 draws ambient air into the hosing 820 via the collection opening 825. The hosing 820 may include any of a variety of tubing, piping, conduits, etc, that are suitable for transporting or communicating samples of air. The pump 810 directs the sample air through the hosing and to the ozone sensor 830, which measures the ozone levels in the sample air. The ozone sensor 830 may be positioned at the skid 20.

The ozone sensor 830 is in electrical communication with the control processor 500 of the system 10. If the ozone level in the sample air is too high, for example the ozone levels are above 0.1 ppm, then the OSHA compliance package 800 in conjunction with the control processor 500 shuts down the system 10 and/or stops the further distribution of the ozonated fluid.

The compliance package 800 may include an electrical controller 850 that may also be inputted or programmed to provide warning signals should a threshold level of ozone may be measured. The control processor 500 may also be programmed to provide such warning signals. For example, the electrical controller 850 may programmed to provide a warning signal or alarm if ozone levels exceed, for example, 0.06 or 0.08 ppm. If the sensor 830 measures these levels, then the compliance package 800 may trigger a warning signal, such as audible or visual alarm or other type of notification. As such, the operator will be warned of the threshold level of ozone and may have the opportunity to correct the system 10 before the ozone levels increase to where the system 10 is shut down.

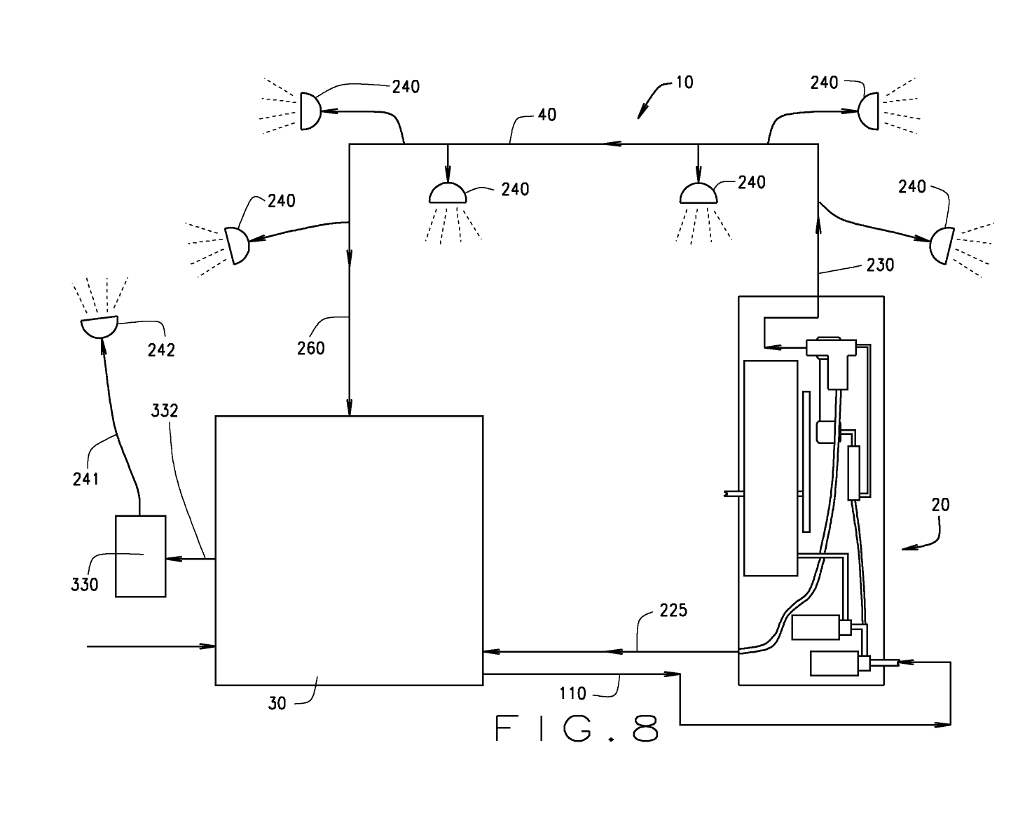

In certain embodiments, as shown in FIG. 8, the tank 30 may include an optional application pump 330 that pumps ozonated fluid directly from the tank 30. The tank 30 is fluidly connected to the application pump 330 via an application pump line 332. The tank 30 may be filled by the tank fill line 225 with the ozonated fluid produced by the skid 20 to form a batch of ozonated fluid in the tank 30 of, for example, hundred gallons or more. The batch is pumped from the tank 30 by the application pump 330 through an application line 241 and to an applicator 242.

It should be understood from the foregoing that, while particular embodiments of the invention have been illustrated and described, various modifications can be made thereto without departing from the spirit and scope of the present invention. Therefore, it is not intended that the invention be limited by the specification; instead, the scope of the present invention is intended to be limited only by the appended claims.

What is claimed:

1. A system for producing and distributing an ozonated fluid, comprising:
a tank for a fluid;
a skid in fluidic communication with the tank to receive the fluid from the tank;
the skid comprising an ozone generator to generate ozone gas, an injector to inject the fluid with the ozone gas to produce an ozonated fluid, a skid output line in fluidic communication with the skid and a distribution network to supply the distribution network with the ozonated fluid, and the skid output line in fluidic communication with the skid and the tank to supply the tank with the ozonated fluid;
the distribution network distributes the ozonated fluid for application; and,
the distribution network is in fluid communication with the tank to return unapplied ozonated fluid to the tank.

2. The system according to claim 1, wherein the skid selectively supplies ozonated fluid to both the distribution network and the tank.

3. The system according to claim 1, further comprising the skid output line in fluidic communication with a tank fill line and a distribution network supply line, wherein the tank fill line fills the tank with ozonated fluid from the skid, and wherein the distribution network supply line supplies the distribution network with the ozonated fluid.

4. The system according to claim 3, wherein the tank fill line includes a tank valve to open and close the tank fill line, and wherein the distribution network supply line includes a distribution network valve to open and close the distribution network supply line.

5. The system according to claim 4, wherein adjusting the tank valve and the distribution network valve controls the amount of fluid passed to the tank fill line and the distribution network supply line.

6. The system according to claim 1, wherein the distribution network comprises one or more applicators in fluidic communication with the distribution network to apply the ozonated fluid.

7. The system according to claim 1, further comprising an inlet pump to supply the skid with fluid and an injector pump to supply the injector with the fluid.

8. The system according to claim 1, wherein an application pump pumps ozonated fluid directly from the tank to an applicator.

9. The system according to claim 1, wherein the tank receives water from a municipal water supply, the tank receives the ozonated fluid from the skid output line of the skid, and the tank receives ozonated fluid from a distribution network return line.

10. The system according to claim 1, wherein the system produces and distributes up to approximately 50 gallons per minute of the ozonated fluid, wherein the ozonated fluid has an ozone concentration of approximately 5 parts per million.

11. The system according to claim 1, wherein an injector pump line supplies the injector with the fluid, a skid return line is in fluidic communication with the injector pump line, and the skid return lines supplies the injector pump line with ozonated fluid that does not enter the skid output line.

12. The system according to claim 1, wherein the injector receives the ozone gas from the ozone generator, wherein the injector is in fluidic communication with the injector pump, the injector pumps supplies fluid to the injector, and the injector injects the ozone gas into the fluid via pressure caused by the injector pump.

13. The system according to claim 1, wherein the system is modular and moveable about a facility.

14. The system according to claim 1, further comprising a dissolved ozone monitoring system, comprising:
a local sensor positioned to measure dissolved ozone levels in the ozonated fluid produced by the skid while the fluid is proximate the skid;
a remote sensor positioned in the distribution network at point of application of the ozonated fluid;
the local sensor in electrical communication with a local monitor; and
the remote sensor in electrical communication with a remote monitor.

15. The system according to claim 14, wherein the local monitor and the remote monitor are housed at the skid.

16. The system according to claim 14, wherein the producing and distributing system comprises a control processor to operate the producing and distributing system, wherein the local monitor and the remote monitor are in electrical communication with the control processor, wherein the control processor modulates the concentration of ozone in the ozonated fluid produced by the producing and distributing system based on the measurements of ozone concentration from the remote sensor in the ozonated fluid at the point of application.

17. The system according to claim 1, further comprising a monitoring assembly to monitor ozone gas levels at a point of application of the ozonated fluid, the monitoring assembly comprising:
hosing, the hosing comprising a collection opening to receive sample air;
an ozone sensor to measure ozone levels in the sample air; and
a pump in fluidic communication with the hosing to transfer the sample air to from the collection opening to the ozone sensor, wherein the ozone sensor measures the ozone levels in the sample air.

18. The system according to claim 17, wherein the monitoring assembly is in electrical communication with a control processor of the system, and the control processor stops the system from distributing the ozonated fluid if the monitoring assembly measures designated levels of ozone gas in the sample air.

19. A system for producing and distributing an ozonated fluid, comprising:
a tank to store a fluid, the tank in fluidic communication with a source of fluid;
a skid to produce ozonated fluid;
a skid supply line fluidly connecting the tank with the skid to supply the skid with the fluid;
the skid comprising an ozone generator to generate ozone gas and an injector to inject the fluid with the ozone gas to produce an ozonated fluid from the fluid;
a skid output line to output the ozonated fluid from the skid;
one or more fluid lines connecting to the skid output line that receive the ozonated fluid from the skid and supply one or more applicators with the ozonated fluid for application, the one or more fluid lines comprising a first valve to open or close the one or more fluid lines; and,
a tank fill line fluidly connecting the tank and to the skid output line to supply the tank with ozonated fluid, the tank fill line comprising a second valve to open or close the tank fill line.

20. A system for producing and distributing an ozonated fluid, comprising:
a reservoir for a fluid;
a skid in fluidic communication with the reservoir to receive the fluid from the reservoir;
an inlet pump to supply the skid with the fluid;
the skid comprising an oxygen concentrator to produce oxygen gas, the oxygen concentrator in supply communication with an ozone generator to generate ozone gas from the oxygen gas, an injector pump to supply an injector with the fluid from the inlet pump, the injector injects the fluid with the ozone gas from the ozone generator to produce an ozonated fluid, a degassing system to remove excess ozone gas from the ozonated fluid, an ozone destruct unit to destroy the excess ozone gas, a reaction vessel to process the ozonated fluid, a skid output line comprising valves for selectively supplying the ozonated fluid to a distribution network or to the reservoir; and,
the distribution network distributes the ozonated fluid to one or more applicators that spray or apply the ozonated fluid.

21. A method for producing and distributing an ozonated fluid, comprising,
providing a reservoir for a fluid; a skid in fluidic communication with the reservoir to receive the fluid from the reservoir; the skid comprising an ozone generator to generate ozone gas and an injector to inject the fluid with ozone gas to produce an ozonated fluid; a distribution network to distribute the ozonated fluid for application; and the distribution network in fluid communication with the reservoir to return ozonated fluid to the reservoir;
providing fluid to the reservoir;
pumping the fluid from the reservoir to the skid;
ozonating the fluid at the skid to produce an ozonated fluid;
modulating the concentration of ozone in the ozonated fluid;
distributing the ozonated fluid via the distribution network;
applying a first portion of the ozonated fluid; and,
returning a second portion of the ozonated fluid to the reservoir.

22. The method according to claim 21, further comprising pumping a mixture of the ozonated fluid and the fluid from the reservoir to the skid, and injecting the mixture with additional ozone.

23. The method according to claim 21, further comprising monitoring ozone gas levels in the ambient air at a point of application of the ozonated fluid, and stopping distribution of the ozonated fluid if ozone gas levels exceed designated levels.

24. The method according to claim 21, further comprising measuring dissolved ozone levels in the ozonated fluid at the application point, and modulating the concentration of ozone in the ozonated fluid at the skid based on the measured dissolved ozone levels at the application point.

25. The method according to claim 21, further comprising filling the reservoir with a batch of ozonated fluid produced by the skid, and pumping the batch of ozonated fluid from the tank by an application pump.

26. A system to monitor ozone levels of an ozonated fluid applied by ozonated fluid producing and dispensing equipment, comprising:
a local sensor positioned to measure dissolved ozone levels in ozonated fluid produced by equipment for producing and dispensing ozonated fluid;
a remote sensor positioned at point of application of the ozonated fluid to measure dissolved ozone levels at the application point;
the local sensor in electrical communication with a local monitor; and,
the remote sensor in electrical communication with a remote monitor.

27. The system to monitor ozone levels according to claim 26, wherein the system communicates the measurements from the remote sensor and the local sensor to a control processor of ozonated fluid producing and dispensing equipment.

28. A system to measure ozone gas levels in ambient air for use with ozonated fluid dispensing equipment, comprising:
hosing, the hosing comprising a collection opening to receive sample air;
an ozone sensor to measure ozone levels in the sample air; and,
a pump in fluidic communication with the hosing to transfer the sample air to from the collection opening to the ozone sensor, wherein the ozone sensor measures the ozone levels in the sample air.

29. The system to measure ozone gas levels according to claim 28, further comprising an electrical controller that signals or initiates a shut-down of ozonated fluid dispensing equipment based on the ozone levels measured by the ozone sensor.

30. The system to measure ozone gas levels according to claim 29, wherein the electrical controller is programmed with a threshold level, and the electrical controller signals or initiates the shut-down of the ozonated fluid dispensing equipment when the ozone levels measured by the ozone sensor exceed the threshold level.
